(19) United States
(12) Patent Application Publication
(10) Pub. No.: US 2011/0287170 A1
(43) Pub. Date: Nov. 24, 2011

(54) METHOD AND APPARATUS FOR APPLYING PARTICULATE

(75) Inventors: Howard E. Colclough, JR., Lynn, MA (US); Alesandro Cucci, Wakefield, MA (US); Kenneth James Farley, Arlington, MA (US); Evans Francois, Newmarket, NH (US); Casper Pronk, Boston, MA (US)

(73) Assignee: Innovent, an unincorporated division of Standex International Corporation, Salem, NH (US)

(21) Appl. No.: 12/743,066

(22) PCT Filed: Dec. 5, 2008
(86) PCT No.: PCT/US08/85691

§ 371 Date: Jun. 2, 2011

Related U.S. Application Data
(60) Provisional application No. 60/992,636, filed on Dec. 5, 2007.

(51) Int. Cl. B05C 19/04; A61F 13/51

(52) U.S. Cl. 427/2.31; 118/308

(57) ABSTRACT

The present disclosure relates to a particulate feeding apparatus for applying particulate, such as super-absorbent materials, to a substrate, such as a fibrous web. The apparatus may include a feeder tube for the powder and a rotary gate valve, including one or more holes, which may then intermittently feed particulate to an eductor or venturi nozzle which is located in an air stream. The rotary valve provides an intermittent supply of particulate to a relatively low pressure zone at the nozzle formed by the venturi action of the passing air stream and the particulate may be distributed precisely where desired. A process for delivering powder to a substrate in precise amounts and distribution patterns is also disclosed.

### CROSS-REFERENCE TO RELATED APPLICATIONS

The present application claims the benefit of U.S. Provisional Application No. 60/992,636 filed on Dec. 5, 2007, the teachings of which are incorporated herein by reference.

### FIELD

This disclosure relates to a method and apparatus for applying a particulate to a substrate and, more particularly, to a method of applying absorbent particulate to a fibrous web for the relatively high speed manufacture of absorbent articles, utilizing a rotary slide gate valve.

### BACKGROUND

Absorbent articles, such as disposable diapers, incontinence pads and the like, may be formed by air-laying fibrous materials on a foraminous surface and depositing super-absorbent materials in particulate or powder form across or throughout the fibrous web. The super-absorbent materials may be directed to certain specific areas of the web where fluids may be concentrated to improve the efficiency of containing and absorbing such fluids. It may be desirable to closely control the application of the super-absorbent materials to only localized areas of the web where they may encounter liquids, due to their relatively higher cost.

Diapers and like absorbent pads may be manufactured at very high production rates, for instance, 200 to 2000 units per minute. In addition, specific patterns of desired absorbency may vary in shape and location depending on the size and intended use of the absorbent pad.

It is thus desirable to provide a particulate metering assembly which has the ability to direct particles in a specific pattern to a specific area in an intermittent fashion, and to do so at a relatively rapid pace.

### SUMMARY

In one exemplary embodiment, the present disclosure comprises a particulate feeding apparatus for applying powder, such as super-absorbent materials, to a substrate, such as a fibrous web. The apparatus comprises a feeder tube for the powder and a rotary gate valve, including one or more holes, to intermittently feed powder to an eductor or venturi nozzle which is located in an air stream. The rotary valve provides an intermittent supply of powder to a relatively low pressure zone at the nozzle formed by the venturi action of the passing air stream and the powder may be distributed precisely where desired.

In a second embodiment, a process for delivering powder to a substrate in precise amounts and distribution patterns is provided wherein a rotary gate valve having one or more openings in a sealing face may be rotated at high speed to provide a supply of powder to a distribution nozzle. The nozzle may be located in a stream of venturi air. The venturi effect of relatively high pressure air passing the end of the nozzle may draw the powder into the air stream for precise distribution to a substrate, such as a fibrous web.

The feeding apparatus of the present disclosure may operate to deliver powder in high speed manufacturing processes that may operate to produce diapers at a rate of about 1200 diapers per minute, utilizing valve actuations at increments of about 0.05 seconds.

### BRIEF DESCRIPTION OF THE DRAWINGS

The features, operation and advantages of the invention may be better understood from the following detailed description of the preferred embodiments taken in conjunction with the attached drawings, in which

FIG. 1A is a cross-sectional view of the particulate feeding apparatus of the present disclosure,

FIG. 1B is an enlarged cross-sectional view of the valve and nozzle of the particulate feeding apparatus of the present disclosure, and

FIG. 2 is an exploded view of the particulate feeding apparatus of FIG. 1, illustrating the rotary gate valve.

### DETAILED DESCRIPTION

Diapers and other personal hygiene products having a capability of containing and absorbing liquids, such as body fluids, may be manufactured at very high production rates, for instance 200-2000 units per minute, by laying a web of a pulp fiber material, such as cellulose, on to a conveyor or forming drum through which air may be drawn. As the understanding of the human anatomy has increased, it has become important to improve the absorbency of fluids in specific areas of the diaper, for instance, more in the front and back of the user as opposed to the crotch. In addition, cost and weight considerations demand that a minimum amount of absorbent material be used.

The use of super-absorbent materials or polymers, such as a polyacrylic acid sodium salt, may greatly increase the absorptive capacity of the fibrous web and may be placed strategically where most fluids may collect. Such super-absorbent materials are generally applied in particulate form to portions of the fibrous web by a spray gun or other feeding apparatus. The particulate form of material may include what is termed as a powder. Accordingly, reference to particulate herein may be understood as a material having a largest cross-sectional diameter of 0.10″ or less and of varying geometries (e.g., round, oval, etc.). In addition, such super-absorbent materials may include those materials that can absorb up to 500 times its weight in a fluid, such as water. It is worth noting that the super absorbent polymers may include other resins. For example, such resins may include polyacrylamide polymers, ethylene-maleic anyhydride copolymer, cross-linked carboxy-methyl-cellulose, poly(vinyl alcohol), cross-linked polyethylene oxide, and/or starch grafted copolymers of poly(acrylonitrile).

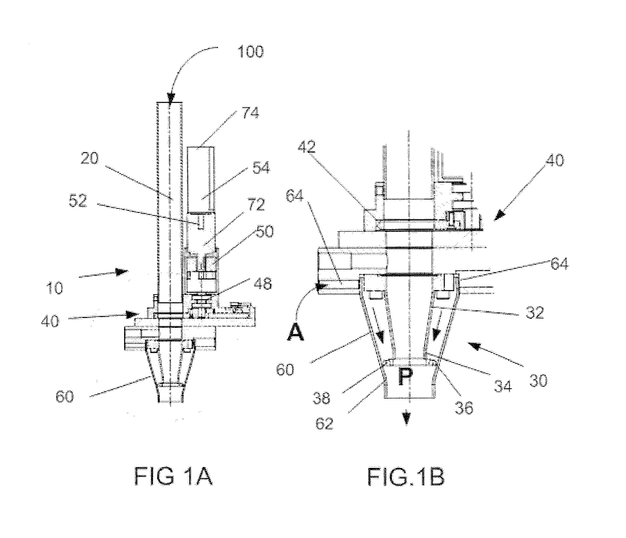

To minimize the amount of super-absorbent material used, it may be important to deliver precise quantities to specific areas of the web via a feeder apparatus. A feeding apparatus 10, according to the present disclosure, is illustrated in FIG. 1A. The feeding apparatus may be positioned adjacent a conveyor or forming drum onto which a fibrous web has been deposited and may direct an intermittent flow of super-absorbent material in powder or particulate form to specific areas and in specific patterns by moving the apparatus relative to the web and by the action of a rotary gate valve 40 which may feed powder to a venturi nozzle assembly 30 (See FIG. 1B).

The feeding apparatus 10 may further comprise a feeder tube 20 which provides particulate material 100 to the valve 40. The particulate may be fed through the tube 20 by gravimetric means, by an auger, by a weight-in loss device, or other apparatus as is known in the art, which may provide a constant supply of particulate to the valve 40. In one exemplary embodiment, the feeder tube 20 may be a 1.5 inch diameter stainless steel tube about 12 inches long.

As shown in FIG. 1A, the valve 40 may comprise a rotating disc assembly 42 driven by a shaft 52 engaged to a motor 54 through a double sealed radial bearing 48 and two Lovejoy shaft couplings 50. The motor may further include a gearbox 72 and motor controller 74.

At the output end of the feeding apparatus 10, a venturi nozzle assembly 30 may be provided to direct the flow of particulate to a fibrous web. The venturi nozzle assembly 30 may comprise an inner nozzle 32 which may be aligned along a common longitudinal axis with the feeder tube 20. The inner nozzle 32 may have a tapered shape in the form of a venturi portion 34 as well as a flared end portion 36 including a plurality of through holes 38. An outer nozzle 60 may be located outside of and concentric with the inner nozzle 32.

The venturi nozzles provide a restriction to the flow of air, A, as it passes between the nozzles, 32 and 60, which may cause an increase in velocity of the air and a drop in pressure, P.

The outer nozzle 60 may have a tapered shape in the form of a venturi portion 62 which fits closely over the flared end portion 36 of the inner nozzle 32. One or more air inlets 64 may be provided at the inlet end of the nozzle 32 to provide a supply of pressurized air to the nozzle assembly 30.

The flow of a stream of air, A, through the nozzle assembly 30 may cause an area of lower pressure, P, to be formed downstream of the flared end portion 36 due to the venturi effect and when rotary gate valve 40 is open, powder 100 may be drawn by suction into the air stream, A, and projected from the nozzle assembly 30 towards a target substrate (not shown).

The plurality of holes 38 may be angled relative to the longitudinal axis of the nozzle assembly 30 to provide a further reduction in pressure at the outlet of the inner nozzle 32 and reduce the area of the projected cone of dispersed powder. Holes 38 having a diameter of about 0.005-0.250 inches with either straight or tapered cross sections and at angles including 0° (or parallel with the longitudinal axis of the nozzle assembly) to 45° from the longitudinal axis of the nozzle assembly 30, including all angles therebetween, are contemplated.

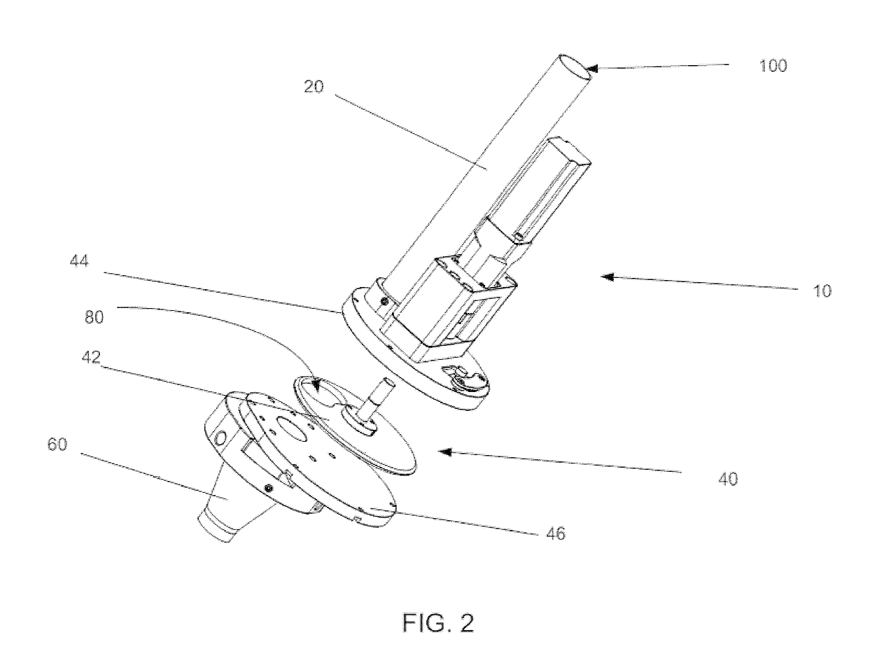

FIG. 2 is an exploded view of the apparatus 10 of FIG. 1 illustrating more clearly the rotary gate valve 40. The valve 40 may comprise a rotating disc assembly 42 and a top 44 and bottom 46 sealing plate. One or more openings 80 may be formed in the rotating disc assembly 42 which when rotated in line with the feeding tube 20 allow powder 100 to be drawn through the one or more openings into the inner nozzle assembly 32 by the low pressure, P, formed by the air stream, A, being forced through the venturi nozzle assembly 30.

By varying the speed at which the rotating disc assembly 42 rotates, it is possible to vary the amount (duration) of powder or particulate 100 directed towards a target substrate such as a web. For instance, the disk 42 may be operated at about 1200 rpm with air supplied at about 90 psi to deliver particulate at a rate of about 5-25 g/diaper to a conveying mechanism producing about 1200 diapers per minute. Thus the valve 40 may operate to open and close in increments as small as, for instance, 0.05 seconds, or in the range of 0.03-0.30 seconds, including all values and increments therein. It may therefore be appreciated that one may therefore operate the apparatus herein where the disk may rotate at 200-2000 rpms and the air supplied may be at pressures of 50-200 psi, including all values and increments therein.

The size, shape and number of openings 80 may be varied to provide different patterns of coverage of the powder 100 onto a given substrate location. For example, one may provide a generally round pattern, an oval pattern, etc. In one exemplary embodiment, the opening 80 may be shaped with respect to its cross sectional area such that the leading edge of the opening may have a lesser or greater cross sectional area than the trailing edge of the opening (e.g. tapered) such that the distribution of the particle stream is varied.

In method form, the present disclosure provides a process to localize the application of a powder or particulate material 100 on a substrate by providing a substrate, such as a fibrous web, providing a particulate, such as a super-absorbent material, to be applied precisely to localized areas of the web. The method includes providing a feeding apparatus 10 noted above, which again includes a feeder tube 20, a driven rotary gate valve 40 and a venturi nozzle assembly 30 to direct the powder onto the web. The venturi nozzle assembly includes inner 32 and outer 60 venturi nozzles which when fed with a supply of pressurized air, A, creates an area of relatively low pressure, P, at the outlet or downstream side of the rotary gate valve 40. Upon rotation of the disk assembly 42 of the valve 40, one or more openings 80 in the disk assembly 42 may align with the feeding tube 20 and the inner venturi nozzle 32 to allow powder to be extracted from the feeding tube 20 and mixed with the air stream, A, which may be directed at the substrate.

Accordingly, the present disclosure provides a relatively high speed and pulsed or intermittent supply of particulate material at selected locations on a substrate, such as a non-woven substrate, which may be used in, e.g., a diaper application. The ability to manufacture and target absorbent particulate, at a selected location, with a selected pattern, and at the speeds noted herein, may therefore provide a relatively more efficient manufacturing operation which may be essential for the formation of products that require relatively high volume production.

While particular embodiments of the present invention have been disclosed, it should be clear to those skilled in the art that various changes and modifications can be made without departing from the scope of the invention.

1. A feeding apparatus for delivering quantities of particulate to specific locations on a substrate, comprising:
a feeder tube, said tube having a longitudinal axis;
a valve, wherein said valve comprises a rotary gate valve; and
a venturi nozzle assembly, said assembly comprising an inner nozzle and an outer nozzle concentric with said inner nozzle, said inner nozzle including a tapered shape and a flared end portion, said end portion including a plurality of through holes.

2. The feeding apparatus of claim 1 wherein said inner nozzle has a longitudinal axis and said inner nozzle longitudinal axis is aligned with said longitudinal axis of said tube.

3. The feeding apparatus of claim 1 wherein rotary gate valve comprises a rotating disc assembly, a top sealing plate and a bottom sealing plate wherein one or more openings are formed in said rotating disc assembly.

4. The feeding apparatus of claim 1 wherein said feeder tube is fed by gravimetric means.

5. The feeding apparatus of claim 1 wherein said plurality of through holes in said inner nozzle flared end portion are angled relative to said inner nozzle longitudinal axis.

6. The feeding apparatus of claim 1 wherein said one or more openings may be round, oval or tapered.

7. The feeding apparatus of claim 1 wherein said rotary gate valve is capable of opening and closing in increments in the range of about 0.03 to about 0.30 seconds.

8. A method for delivering quantities of particulate to specific locations on a substrate, comprising:
providing a feeding apparatus, wherein said apparatus comprises a feeder tube, said tube having a longitudinal axis; a valve, wherein said valve comprises a rotary gate valve including a rotating disc assembly having one or more openings; and a venturi nozzle assembly, said assembly comprising an inner nozzle and an outer nozzle concentric with said inner nozzle, said inner nozzle including a tapered shape and a flared end portion, said end portion including a plurality of through holes;
providing a quantity of particulate material to said feeder tube;
rotating said rotating disc assembly;
providing a source of air to said venturi nozzle assembly;
drawing said particulate from said feeder tube through said openings in said rotating disc assembly and through said venturi nozzle assembly; and
projecting said particulate onto said substrate.

9. The method of claim 8 wherein said particulate comprises a super-absorbent material.

10. The method according to claim 8 wherein said substrate comprises a fibrous web.

11. The method of claim 8 wherein said particulate is applied in a pattern to said substrate.

12. The method of claim 8 wherein said particulate is intermittently applied to said substrate.

13. The method of claim 8 wherein said rotary gate valve opens and closes in increments in the range of about 0.03 to about 0.30 seconds.

14. The method of claim 8 wherein said feeder tube is fed by gravimetric means.

15. The method of claim 8 wherein said plurality of through holes in said inner nozzle flared end portion are angled relative to said inner nozzle longitudinal axis.

16. The method of claim 8 wherein said one or more openings in said rotating disc assembly may be round, oval or tapered.

17. The method of claim 8 wherein said inner nozzle has a longitudinal axis and said inner nozzle longitudinal axis is aligned with said longitudinal axis of said tube.
